(19) United States
(12) Patent Application Publication
(10) Pub. No.: US 2006/0024674 A1
(43) Pub. Date: Feb. 2, 2006

(54) Method for conducting the parallel identification of different nucleic acids

(75) Inventors: Ronald Barten, Erlangen (DE); Dirk Kuhlmeier, Munchen (DE); Anja Weiland, Poxdorf (DE); Hans Kosak, Bonn (DE); Jorg Hassmann, Erlangen (DE)

Correspondence Address: KANESAKA BERNER AND PARTNERS LLP, SUITE 300, 1700 DIAGONAL RD, ALEXANDRIA, VA, 22314-2848, US

(73) Assignee: November Aktiengesellschaft, Erlangen (DE)

(21) Appl. No.: 10/534,711

(22) PCT Filed: Nov. 13, 2003
(86) PCT No.: PCT/EP03/12694

§ 371 Date: Jun. 23, 2005

(30) Foreign Application Priority Data
Nov. 14, 2002 (DE) 102 53 337.7

(51) Int. Cl. C12Q 1/68; C12P 19/34

(52) U.S. Cl. 435/6; 435/91.2

(57) ABSTRACT

The invention relates to a method for conducting the parallel identification of different nucleic acids S. When nucleic acids S are present, primers are lengthened each of which having an intermediate segment z. The intermediate segment z is amplified by using a second primer pair and is specifically identified by hybridizing with a probe So.

The invention relates to a method for detecting different nucleic acids in parallel and to a kit which is suitable for implementing the method.

EP 0 332 435 B2 discloses a method of this nature. In this method, a diagnostic primer, which is essentially complementary to a diagnostic moiety of a nucleotide sequence to be detected, is only extended in a primer extension reaction if the terminal nucleotide of the diagnostic primer is complementary to the corresponding nucleotide in the diagnostic moiety. The product of the extension is used as the template in an amplification reaction which is carried out using a primer pair. The presence or absence of the nucleotide sequence to be detected is established by the presence or absence of an amplification product.

The amplification products can be detected by means of gel electrophoresis. For this, it is necessary for it to be possible to identify the amplification products specifically by their length or by means of a specific label. For this reason, when a number of detection reactions are to be carried out in parallel, the method only makes it possible to detect a very limited number of different amplification products. Another disadvantage of this method is that it is relatively elaborate, particularly when a large number of detections are being performed. The precipitation of amplification products which are labeled, for example radioactively, is cited as another detection method. In this method, it is only possible to detect in parallel as many amplification products as can be differentiated from each other on the basis of their specific labeling.

WO 00/58516 discloses a method for detecting nucleic acids in parallel. In this method, a solution which contains nucleic acids which are potentially to be detected is brought into contact with a multiplicity of primers. The primers in each case possess a 3′-oriented first segment, which is specific for a nucleic acid to be detected, and a 5′-oriented second segment which can be unambiguously identified by hybridization. The primers are brought into contact with the nucleic acids to be detected under conditions under which specific hybridization can take place. Hybridized primers are in each case extended by a specifically labeled nucleotide in an extension reaction. This thereby makes it possible to differentiate between nucleic acids which are to be detected and which differ in the nucleotide which is complementary thereto. The primer extension products are brought into contact with a template on whose surface oligonucleotides which are complementary to the second segments are immobilized at defined positions. Following hybridization, the extended primers are detected specifically by their location on the template and their specific labeling. The method can be used for detecting nucleic acids which only differ in one nucleotide. A disadvantage is that the method is not very sensitive. It is also disadvantageous that specifically labeled nucleotides are required.

Brownie, J. et al., Nucleic Acids Research, volume 25, No. 16 (1997), pages 3235 to 3241 discloses a method for preventing the formation of primer dimers in a PCR. This method uses primers which possess an appendage, consisting of an additional nucleotide sequence, at their 5′ ends. These primers are present, in the PCR, at a concentration which is so low that they only take part in early cycles of the PCR. In subsequent PCR cycles, a single primer possessing the sequence of the appendage is used for the amplification. In the case of amplification products formed from primer dimers, the sequence of the appendage leads to complementary sequences, which are derived from the sequence of the appendage, of an individual amplification product hybridizing with each other, thereby forming a type of “pan handle” structure. The hybridization prevents further primers from annealing to the appendage, with this thereby avoiding the production of unwanted PCR products. The PCR products are detected by gel electrophoresis. This method is elaborate and, in the case of reactions which are carried out in parallel in a PCR, only allows a very limited number of PCR products to be detected.

U.S. Pat. No. 5,849,544 discloses a method for detecting a nucleic acid. In this method, a trapping probe is immobilized on a vessel wall. The nucleic acid to be detected is amplified, for example by means of a PCR. In connection with this, a label to be detected, for example a biotin group, is also incorporated into the amplification product. The trapping probe possesses a nucleic acid sequence which can hybridize with at least a part of the amplified target nucleotide sequence. The amplified target nucleotide sequence is brought into contact with the trapping probe under conditions which allow the trapping probe to bind to the amplified target nucleotide sequence. The bound amplified target nucleotide sequence can be detected using the labeling substance.

Jenison, R. et al., Biosensors and Bioelectronics 16 (2001), pages 757 to 763 discloses a chip which contains trapping probes, which are specific for particular viral sequences, on a surface. The chip enables specific products of a multiplex PCR to be detected. The multiplex PCR uses a mixture of different primers which are in each case specific for a sequence to be amplified. PCR products which are bound to the trapping probes are detected optically by means of a change in the color of a light which is reflected from the surface, with this change being brought about by the binding and an enzyme-catalyzed reaction. A disadvantage of this method is that, as a result of using specific primers for each of the sequences to be detected, these sequences can be amplified with differing efficiencies.

U.S. Pat. No. 5,871,918 discloses a method for electrochemically detecting nucleic acids using an immobilized trapping probe. Following hybridization of the nucleic acid to be detected to the trapping probe, the hybridized DNA is brought into contact with a transition metal complex which is able to oxidize a predetermined base in the trapping probe. The redox reaction is detected electrochemically and compared with a redox reaction of the single-stranded probe. If the reaction rates which are determined in this connection differ from each other, this then indicates the assistance of a hybridization.

A disadvantage of the methods disclosed in U.S. Pat. No. 5,849,544, U.S. Pat. No. 5,871,918 and Jenison et al. is that, for the purpose of detecting different nucleic acids in parallel under uniform conditions, it is in each case necessary to determine a sequence for a trapping probe which enables one of the nucleic acids to be detected, or a PCR product of these nucleic acids, to bind specifically under these conditions. The greater the number of nucleic acids which are to be detected alongside each other, the greater is the input required for determining the sequences for the trapping probes.

DE 199 34 084 A1 discloses a method for labeling and characterizing DNA fragments. This method involves carrying out a PCR. The PCR uses three primers, two of which are sequence-specific oligonucleotide primers. One of these oligonucleotide primers is provided with an adaptor sequence which is homologous with the oligonucleotide sequence of the third primer. The third primer exhibits a labeling substance and is used for generating amplification products which possess a label. The disadvantage of this method is that, when it is carried out simultaneously using several DNA fragments which are to be characterized under uniform conditions, the different DNA fragments can be amplified with differing efficiencies owing to the different binding kinetics of the primers.

The object of the present invention is to provide a method and a kit for detecting a nucleic acid, which method/kit avoids the disadvantages of the prior art. In particular, the method should be possible to carry out using simple means and should exhibit a high degree of sensitivity. The method should, in particular, make it possible to detect a large number of different nucleotide sequences in parallel and under uniform conditions and, in particular, in a common reaction mixture.

This object is achieved by means of the features of claims 1 and 31. Expedient embodiments ensue from the features of claims 2 to 30 and 32 to 38.

The invention envisages a method for detecting different nucleic acids A in parallel, comprising the following steps:
a) providing in each case one first primer pair which is suitable for carrying out a PCR together with one of the nucleic acids A and which contains a first primer (P1) and a second primer (P2),
with the first primer (P1) exhibiting a 5′-terminal first constituent segment (c1) and a 3′-terminal second constituent segment (c2) and the second primer (P2) exhibiting a 5′-terminal third constituent segment (c3) and a 3′-terminal fourth constituent segment (c4),
with the sequences of the second constituent segment (c2) and the fourth constituent segment (c4) being selected such that the second constituent segment (c2) can hybridize specifically, under defined first conditions, with a predetermined first segment of the one of the nucleic acids A, and be enzymically extended, and the fourth constituent segment (c4) can hybridize specifically, under defined second conditions, with a predetermined second segment of a nucleic acid A′ which is complementary to the one of the nucleic acids A, and be enzymically extended, and
with in each case an intermediate segment i, which connects the first constituent segment (c1) to the second constituent segment (c2) and is specific for the second constituent segment (c2), or an intermediate segment i, which connects the third constituent segment (c3) to the fourth constituent segment (c4) and is specific for the fourth constituent segment (c4), being provided,
with the first (P1) or second primers (P2) of the first primer pairs in each case differing in the intermediate segment i and in the second constituent segment (c2) or fourth constituent segment (c4) which is arranged in connection thereto, with each of the second (c2) or fourth constituent segments (c4) being specific for precisely one of the nucleic acids A,
b) bringing the different nucleic acids A, or the nucleic acids A′ which are complementary thereto, into contact with the first primer pairs in a solution and carrying out a first primer extension reaction in which the first primers (P1) are extended, under the first conditions, or the second primers (P2) are extended, under the second conditions, at least once and at least so far that the respective other primers (P2, P1) of the first primer pairs are able to bind specifically, under the first or second conditions which are required for their specific hybridization, to in each case one first primer extension product which is formed in this connection,
c) carrying out a second primer extension reaction in which the first primer extension products in each case serve as a template and the respective second (P2) or first primers (P1) are extended, under the first or second conditions which are required for their specific hybridization with the respective first primer extension products, with the formation of in each case one second primer extension product,
d) providing in each case one second primer pair which in each case contains a third primer (P3) and a fourth primer (P4) and which is suitable for carrying out a PCR together with the respective second primer extension products,
with the sequences of the third primer (P3) and fourth primer (P4) being in each case selected such that the third primer (P3) can in each case hybridize specifically, under defined third conditions, with a sequence which is complementary to the first constituent segment (c1), and be enzymically extended, and the fourth primer (P4) can in each case hybridize specifically, under defined third conditions, with a sequence which is complementary to the third constituent segment (c3) and be enzymically extended,
e) bringing the second primer extension products into contact with the respective second primer pairs and carrying out a PCR, with in each case the intermediate segment i, or an intermediate segment i′ which is complementary thereto, being amplified with the formation of third primer extension products,
f) providing in each case one immobilized probe (Pr) for each nucleic acid A to be detected, with the probe (Pr) being in each case able to hybridize specifically, under defined fourth conditions, with one of the intermediate segments i or one of the intermediate segments i′ which are complementary thereto,
g) bringing the probes (Pr) into contact with the third primer extension products under the fourth conditions, and
h) detecting the third primer extension products which bind, or are bound, to the probes (Pr).

The first, second, third and fourth conditions comprise, for example, particular temperatures or concentrations, for example of a primer. The first and second conditions are preferably identical. The first primer extension reaction in accordance with step b and the second primer extension reaction in accordance with step c can be carried out simultaneously. The first and the third constituent segments are selected such that they essentially do not hybridize with the nucleic acid A or the nucleic acid A′, which is complementary thereto, under the first or second conditions. Furthermore, the first and third constituent segments are preferably selected such that they do not form any secondary structures, such as hairpin loops, which interfere with the method and have similar melting temperatures, a length of from 16 to 28 nucleotides, no complementarity to each other, in particular at the 3′ ends, a balanced GC ratio and no GC-rich regions at the 3′ end. Criteria for selecting the sequences are disclosed in the prior art, for example in Michael A. Innis, David H. Gelfand and John J. Sninsky, PCR Applications: Protocols for Functional Genomics, Academic Press, San Diego, Calif., USA (1999).

In one embodiment of the method, the first and third constituent segments have the same length or differ in their length by at most 20%. This makes it possible to ensure that the specific annealing temperatures of the third primer, which hybridizes with the sequence which is complementary to the first constituent segment, and of the fourth primer, which hybridizes with the sequence which is complementary to the third constituent segment, are relatively close to each other. The efficiency with which the PCR according to step e can be carried out depends directly on the closeness of the annealing temperatures.

The probe can in each case be a nucleic acid or an analog of a nucleic acid, such as PNA. In this connection, an analog of a nucleic acid is understood as being any structure which can hybridize specifically with the intermediate segment i or the intermediate segment i′, which is complementary thereto. The probes can be immobilized, for example at a membrane. The third primer extension products which bind, or are bound, to the probes can be identified by the position in which in each case one probe is immobilized being in each case known and the site of the hybridization being detected. The probes can also be constituents of a chip. In this connection, a chip is understood as being a solid, rigid surface on which one of the probes is in each case immobilized at a defined position. Other probes can in each case be immobilized at other defined positions on the surface. In this case, too, the third primer extension product which is hybridized with the respective probe can be identified by determining the site of the hybridization.

In the method, and when the nucleic acid A is present, first or second primers which contain the intermediate segment i are extended. The fact that the intermediate segment i is specific for the second or fourth constituent segment means that a particular intermediate segment i can be unambiguously assigned to a particular second or fourth constituent segment. The intermediate segment i, or the intermediate segment i′ which is complementary thereto, is specifically amplified using third and fourth primers. As a result of hybridizing the intermediate segment i, or the intermediate segment i′ which is complementary thereto, with one of the probes in step g, extension products of the third or fourth primers are bound specifically to one of the probes and can be detected there during or after the binding.

As a result of the fact that each intermediate segment i can be assigned accurately to a second or fourth constituent segment, and each second or fourth constituent segment is specific for precisely one of the nucleic acids A, it is possible to assign each intermediate segment i, or each intermediate segment i′ which is complementary thereto, to one of the nucleic acids A. By means of specifically detecting primer extension products which contain the intermediate segment i or the intermediate segment i′, it is possible to determine whether nucleic acids A which hybridize specifically with the second or fourth constituent segments are present in the solution.

The particular advantage of the method according to the invention is that an almost unlimited number of specific labels for primers can be made available by means of the intermediate segment i. This thereby makes it possible to specifically detect a large number of nucleic acids in parallel. The exponential amplification of the intermediate segment i, or of the intermediate segment i′ which is complementary thereto, which is achieved by the PCR in step e ensures a high degree of sensitivity.

It has thus far been possible to detect a multiplicity of nucleic acids in parallel by means of amplifying these nucleic acids in parallel in a PCR mixture, i.e. what is termed a multiplex PCR. In this method, primer dimers can be formed, and the desired PCR reactions can thereby be inhibited, owing to the high total concentration of primers which are required. Using multiplex PCR to detect a multiplicity of nucleic acids in parallel has therefore thus far been restricted to using a few primer pairs to detect a few nucleic acids. It is therefore particularly advantageous, when using the method according to the invention to detect a multiplicity of different nucleic acids A in parallel, that the primer pair which is in each case the first can be provided in a concentration which is so low that the concentrations of the first primer pairs do not cause any interference.

When using multiplex PCR to detect a multiplicity of nucleic acids in parallel, the nucleic acids have thus far usually been amplified to differing degrees. When amplification occurs to differing degrees, the quantities of the amplification products which are produced can differ from each other such that, while the lowest quantity which is required for the detection has still not been produced in the case of individual nucleotide sequences, other nucleotide sequences can already be detected with ease. The reason for the difference in the degree of amplification can be that, because of the exponential amplification in the PCR, even small differences in the efficiencies of the different primers have a powerful effect on the quantities of the products which are formed. The efficiencies of the primers are determined, in particular, by the lengths of the primers, and the binding kinetics with which the primers bind to the nucleic acid to be detected. However, due to the different sequences of the nucleic acids to be detected, these small differences in efficiency are almost inevitably present in a multiplex PCR. The method uses specific primers of differing sequence and frequently also of differing length. The present invention can overcome this disadvantage by the first and second primer extension reactions having to be carried out only once or a few times. Because an exponential amplification can be dispensed with in this connection, different efficiencies between different first primer pairs in each of the first and second primer extension reactions, in connection with detection reactions which are carried out in parallel, only have a negligibe effect on the quantities of the third primer extension products. The second primer pair can be configured independently of the sequence of the nucleic acid A to be detected. As a result, it is possible to provide second primer pairs of uniform efficiency in the PCR. For example, all the first primer pairs can, for this purpose, contain a first primer having a standardized first constituent segment and a second primer having a standardized third constituent segment. This thereby provides two universal primer binding sites which are the same for all the different nucleic acids A which are to be detected. This thereby makes it possible to provide, in step d, a second primer pair which is common to all the nucleic acids A to be detected and to use it to carry out a PCR in step e.

When several different nucleic acids A are being detected in parallel, another advantage of the method is that the primers can be designed such that the detections can be carried out under uniform conditions. It is furthermore advantageous that, once they have been determined, conditions for hybridizing the intermediate segment i, or the intermediate segment i′ which is complementary thereto, with the probe can be used for different detection reactions. For this, the intermediate segment i in different first or second primers can be combined with different second or fourth constituent segments. A chip possessing different immobilized probes can, for example, be prepared in this way. The sequences of the probes and of the intermediate segments i can be used to optimize the chip for the inventive method, with the chip then being used for detecting different nucleic acids A.

The first primer extension reaction and the second primer extension reaction are preferably carried out as PCRs. As a result, these primer extension reactions can be carried out using enzymes and nucleotides which are required for implementing the PCR in step e. This simplifies the method since enzymes and nucleotides only have to be added once.

It has furthermore proved to be advantageous if the first primer extension reaction and/or the second primer extension reaction and/or PCR(s) is/are carried out under what are termed hot start conditions. Under these conditions, the temperature of the reaction mixture is increased and care is taken to ensure that a DNA polymerase which is employed does not extend the first, second, third and/or fourth primers until the temperature in the reaction mixture has at least reached the temperature which is required for these primers to be specifically annealed. This can, for example, be ensured by the DNA polymerase for the first and/or second primer extension reaction and/or PCR not being added to the respective reaction mixture until after this temperature has been reached or by using a polymerase which has to be heated in order to be activated. This thereby prevents nonspecifically binding first, second, third and fourth primers from being extended.

In an advantageous embodiment of the method, the first primer extension reaction is carried out, under the first conditions, and/or the second primer extension reaction is carried out, under the second conditions, at most 10 times, preferably at most 5 times, in particular at most 2 times. This does not mean that the first and/or second primer extension reaction does not in all take place more frequently than 10, 5 or 2 times but, instead, that it is only carried out at most 10, 5 or 2 times under the first and/or second conditions which are favorable for a specific and efficient first and/or second primer extension reaction. Even when the first and/or second primer extension reaction also takes place subsequently under the third conditions, the third conditions can, by the sequences of the first (c1) and third constituent segments (c3) being configured appropriately, be selected such that they are unfavorable for the first and/or second primer extension reaction such that, in all, a low number of first and/or second primer extension reactions takes place. The result of there being a low number of first and/or second primer extension reactions is that, when a multiplicity of detection reactions are being carried out in parallel in a reaction mixture, the fact that the first and/or second primer extension reactions is/are proceeding with different efficiencies has hardly any effect on the quantity of the third primer extension products which are formed.

It has proved to be particularly advantageous if the sequences of the first constituent segment and third constituent segment are selected such that the third conditions can be so stringent that the second constituent segment does not significantly hybridize, under the third conditions, with the first segment of the one of the nucleic acids A and the fourth constituent segment does not significantly hybridize, under the third conditions, with the second segment of the nucleic acid A′ which is complementary to the one of the nucleic acids A. This makes it possible for the reaction to be carried out in a mixture which contains the first, second, third and fourth primers from the outset. The first and second primer extension reactions can then be terminated merely by increasing the stringency, e.g. by increasing the temperature, and it is only the third and fourth primers which are still extended in the PCR even though the first and second primers are still present in the mixture.

In one embodiment of the method, the sequences and concentrations of the first, second, third and fourth primers are selected such that the specific annealing temperatures of the third primer, which hybridizes with the sequence which is complementary to the first constituent segment, and of the fourth primer, which hybridizes with the sequence which is complementary to the third constituent segment, are in each case at least 5° C. higher than the in each case higher annealing temperatures of the second constituent segment, which hybridizes with the first segment of one of the nucleic acids A, and of the fourth constituent segment, which hybridizes with the second segment of the complementary nucleic acid A′. As a result of the annealing temperatures differing by at least 5° C., the first or second primer extension reaction can reliably be prevented from taking place, when the PCR in accordance with e is carried out, by increasing the stringency, e.g. by increasing the temperature.

Step e can be carried out in the solution. It is not necessary to remove the first primer extension products from the solution and transfer them to another solution. At least steps a to e, in particular steps a to h, are preferably carried out in a closed vessel which is not opened between the steps. This thereby makes it possible to avoid result-falsifying contaminations. In addition, using a closed vessel simplifies the management and automation of the method.

In one advantageous embodiment of the method, the concentration, in the solution, of the first or second primer containing the intermediate segment i is selected to be so low that this primer does not significantly inhibit a hybridization of the probe with the respective intermediate segment i, or with the intermediate segment i′ which is complementary thereto, of the third primer extension products in step g. “Does not significantly inhibit” means that the hybridization in step g takes place to an extent which is adequate for the detection in step h. The features mentioned make it possible to largely prevent such a primer from competing, in step g, with the extension products of the third and fourth primers for binding to the probe. It is also possible to largely prevent the hybridization of the probe with the intermediate segment i′ which is complementary to the intermediate segment i being inhibited by hybridization of the intermediate segment i′ with this primer. This simplifies the method considerably. It is not necessary to remove an excess of the intermediate segment i-containing first or second primers. The low concentration also makes it essentially possible to prevent a hybridization of one of said primers with the probe inducing a signal which is in actual fact used for the detection. In addition, a low concentration of the first primer pair can prevent the formation of dimers composed of first and second primers. When several nucleic acids A are being detected in parallel in a mixture, it makes it possible to use a large number of different first primer pairs without exceeding, in the total concentration of these primer pairs, the concentration range for primers which is favorable for primer extension reactions.

The concentration, in the solution, of the in each case first primer pair is preferably set to be from 0.001 to 0.1 μmol/l. It is advantageous if the ratio of the concentrations of the in each case first primer pair to the in each case second primer pair is less than 1:10, preferably less than 1:100, particularly preferably less than 1:1000. The lower the ratio is, the less does the intermediate segment i of the first or second primer compete with the extension products of the third or fourth primers for binding to the probe or compete with the probe for binding to the intermediate segment i′ which is complementary to the intermediate segment i.

The second primer pairs can be added to the solution prior to the first primer extension reaction. This simplifies the method. As a result, no pipetting steps are required between the first and second primer extension reactions. The implementation of the first or second primer extension reaction can be controlled exclusively by way of the temperature.

In step e, the third primer or the fourth primer is preferably extended more frequently than is the respective other primer of the second primer pair. This can be achieved, for example, by in the second primer pair which is provided in step d, the third primer or the fourth primer being present in what is preferably a 2- to 5-fold excess as compared with the respective other primer which is present in the primer pair. Under these asymmetric conditions, the extension product which binds to the probe can be selectively formed in excess as compared with the other extension product of the third or fourth primer. In this way, it is possible to markedly reduce a hybridization of the extension products of the third and fourth primers which competes with the hybridization with the probe, such that a larger proportion of the third primer extension products binds to the probe. This can improve the sensitivity of the detection or make it possible to obtain a stronger signal in connection with the detection. It furthermore makes the method more efficient and economic because, in each case, only one of the two third primer extension products which are formed is required for the detection. A further advantage is that the PCR products which are formed do not have to be denatured, in order to permit hybridization with the probes, before carrying out step g because only a part of the probe-binding intermediate segments i, or intermediate segments i′ which are complementary thereto, is present hybridized with its counterstrand which is formed in the PCR in accordance with e. If the denaturation is dispensed with, sequences which are folded back on themselves and which are fortuitously present are also frequently not denatured. As a result, these sequences cannot compete with the intermediate segments i, or the intermediate segments i′ which are complementary thereto, for specific hybridization to the probes. This increases the sensitivity and specificity of the detection. If the denaturation which normally takes place by heating does not occur, electrodes which are used for an electrochemical detection are also, for example, subjected to less stress and as a result exhibit greater durability.

A multiplicity of first primer pairs, whose first primers exhibit an in each case identical or almost identical first constituent segment and/or whose second primers exhibit an in each case identical or almost identical third constituent segment, and whose second constituent segment or fourth constituent segment is in each case specific for precisely one of the nucleic acids A, can be added to the solution. Constituent segments are almost identical when the same primers can hybridize with them. This makes it possible to design in each case all third, and in each case all fourth, primers uniformly such that only one second primer pair is required. This considerably simplifies the method.

The sequences of the first, second, third and fourth primers can be selected such that they do not form any primer dimers and/or do not hybridize with themselves or with each other in the method. In the PCR, the formation of primer dimers leads to the production of the desired product, e.g. the third primer extension products, being reduced. Brownie, J. et al., Nucleic Acids Research, Volume 25, No. 16 (1997), pages 3235 to 3241 discloses possibilities for suppressing formation of primer dimers. Furthermore, first primers should not be able to hybridize with second primers, nor third primers with fourth primers, nor first primers with first primers, nor second primers with second primers, nor third primers with third primers, nor fourth primers with fourth primers, or other primers, under the conditions of the method. This increases the efficiency of the method. It is furthermore advantageous for increasing efficiency if the sequences of the intermediate segments i are selected such that neither they themselves, nor the intermediate segments i′ which are complementary thereto, hybridize, in the method, with themselves or with the first, second, third or fourth constituent segments or their complementary sequences.

The sequences of the intermediate segments i are preferably selected such that hybrids of the intermediate segments i with nucleic acid strands which were in each case completely complementary thereto would have melting temperatures which are essentially identical, in particular, lying in a temperature range of 5° C. This simplifies step g because the fourth conditions are essentially identical for all the third primer extension products. As a result, in step g, all the third primer extension products can bind simultaneously to the probes which are in each case specific for them.

The method is also suitable for specifically detecting one of the nucleic acids A in the presence of another nucleic acid which only differs from the one of the nucleic acids A in one first base which is contained in the one of the nucleic acids A. The nucleic acid A and the other nucleic acid can, for example, be polymorphic nucleic acids. In this connection, it is advantageous if the sequences of the first or second primers are selected such that the respective base of the second or fourth constituent segment, which base is complementary to the first base or a second base, which is complementary thereto, of a complementary nucleic acid A′, is located at the 3′ end, or in the vicinity of the 3′ end, of the in each case first or second primer. In this connection, “in the vicinity” means, in particular, that at most 3 nucleotides are present between the respective base and the end. In this connection, the first or second conditions of the first or second primer extension reaction can be selected such that a primer which contains a base which is not complementary is not extended in the first or second primer extension reaction.

The specificity of the method can be further increased by the second or fourth constituent segments containing a base which is not complementary to a third base, which corresponds to it in its position, in the first segment of the one of the nucleic acids A or in the second segment of the nucleic acid A′ which is complementary to the one of the nucleic acids A. A base in the second or fourth constituent segment corresponds in its position to the third base if the third base were to be located opposite it when the second or fourth constituent segment hybridized with the first or second segment. When detecting polymorphic nucleic acids, the noncomplementary base in the second or fourth constituent segments results in the case of a hybridization with another, nonspecific nucleic acid, in two mispairings being present whereas only one mispairing is present in the hybridization with the specific nucleic acid A. A mispairing is understood as being a pairing of noncomplementary bases. A hybridized primer which is only mispaired once is nevertheless frequently extended whereas a primer which is mispaired twice is not extended, in particular because a hybrid which contains two mispairings is relatively unstable.

The respective sequences of the first, second, third and fourth primers, and of the probe, can be selected such that in each case the first, in each case the second, in each case the third and/or in each case the fourth conditions for detecting the different nucleic acids A are identical. A parallel detection of the different nucleic acids A in one reaction mixture is rendered possible if in each case all the first, in each case all the second, in each case all the third and in each case all the fourth conditions are identical. The sequences should be selected such that specific hybridizations, without cross hybridizations, take place under said conditions.

The method can be made particularly efficient if the probes are in each case immobilized on an electrode or in its immediate vicinity. The detection in accordance with step h can then be effected by detecting a change, which is determined by the hybridization, in an electrical property at the electrode. In this connection, “in the immediate vicinity” means that the respective probe is immobilized sufficiently close to the electrode for it still to be possible to detect hybridizations with the probe electrically at the electrode and to assign them to the probe.

The electrodes can also, independently of a detection which is based on a change in an electrical property, be used for enriching the third primer extension products at the probe by means of electrostatic attraction. “In the immediate vicinity” then means that the probe is arranged in relation to the electrode such that such an enrichment is possible. The electrode can furthermore also be used to remove nonspecifically bound third primer extension products from the probe by means of electrostatic repulsion. The detection in step h can also be effected by detecting a change, which is determined by the hybridization, in an fluorescent-optical property.

The change in the electrical property can be a change in a redox property, in particular in association with the oxidation of guanine or adenine residues in the third primer extension products, in an impedance or in a conductivity, which change is measured using the respective electrode. The redox property can be a redox potential whose change is detected by electrochemical conversion of the third primer extension product which is bound to the probe, e.g. by means of differential pulse voltametry or chronopotentiometric stripping analysis. For example, the binding of the third primer extension product to the probe can be detected electrochemically by oxidation of its guanine and/or adenine residues. A carbon-containing electrode or a metal electrode, in particular a gold electrode, can be used as the electrode.

The third and/or fourth primer can exhibit a label which can be detected fluorescent-optically at the electrode, or electrically or electrochemically by means of the electrode and which is preferably redox-active. The label can be directly or indirectly detectable. A label which is or possesses a specific affinity molecule is, for example, detectable indirectly. A specific affinity molecule is a molecule to which a counter molecule binds with a high degree of specificity and affinity. The affinity molecule can, for example, be biotin or a hapten and the corresponding counter molecule can be streptavidin or an antibody. The counter molecule can, for example, be conjugated with a fluorescent dye, a redox-active molecule or an enzyme. The enzyme can be an enzyme which can convert a substrate such that the reaction product can be specifically detected electrochemically or optically. The enzyme can, for example, be a phosphatase which can be detected electrochemically by means of the enzymic conversion of naphthyl phosphate. If the label is to be detected directly, it can exhibit an osmium complex, a nanogold particle, a cysteine, ferrocenyl, daunomycin, benzoquinone, naphthoquinone, anthraquinone or p-aminophenol group, a dye, in particular indophenol, thiazine or phenazine, or a fluorescent dye, in particular 6-FAM, HEX, TET, Cy3, Cy5, IRDye™700, IRDye™800, Biodipy, fluorescein, Joe, Rox, TAMRA or Texas Red. Oligonucleotides which are labeled with the abovementioned fluorescent dyes can be obtained from the company Thermo Hybaid, Sedanstrasse 18, D-89077 Ulm, Germany.

Preference is given to using a multiplicity of different probes which are complementary to the intermediate segments i or to the intermediate segments i′ which are complementary thereto, each of which probes is bound to, or in the immediate vicinity of, a separate electrode such that it is possible to deduce the presence of a specific nucleic acid A from a signal at a specific electrode. It is possible to use a multiplicity of electrodes which are arranged on a surface, in particular an electrode chip, so as to be individually bonded or bondable. In this connection, an electrode chip is understood as being a small plate which possesses electronic microstructures and which does not necessarily consist of semiconductor material. The smaller the surface is, the smaller is the volume of the solution which is required for the detection.

An RNA can be detected indirectly by transcribing it into a DNA and then detecting the DNA as nucleic acid A. The transcription can be effected using the “reverse transcriptase” enzyme.

The invention also relates to a kit for carrying out a method, as claimed in one of the preceding claims, for detecting a multiplicity of different nucleic acids A in parallel, with the kit comprising:
a) for each nucleic acid A to be detected, in each case one first primer pair which is suitable for carrying out a PCR together with the nucleic acid A and which contains a first primer and a second primer,
with the first primer exhibiting a 5′-terminal first constituent segment and a 3′-terminal second constituent segment and the second primer exhibiting a 5′-terminal third constituent segment and a 3′-terminal fourth constituent segment,
with the sequences of the second constituent segment and the fourth constituent segment being selected such that the second constituent segment can hybridize specifically, under defined first conditions, with a predetermined first segment of the nucleic acid A which is in each case to be detected, and the fourth constituent segment can hybridize specifically, under defined second conditions, with a predetermined second segment of a nucleic acid A′ which is complementary to the nucleic acid A which is in each case to be detected, and
with an intermediate segment i, which connects the first constituent segment to the second constituent segment and is specific for the second constituent segment, or an intermediate segment i, which connects the third constituent segment to the fourth constituent segment and is specific for the fourth constituent segment, being provided, and
b) for each nucleic acid A to be detected, in each case a second primer pair containing a third primer and a fourth primer, which pair is suitable, together with a primer extension product which can be generated, using the first and second primers, when the nucleic acid A which is in each case to be detected is present, for carrying out a PCR, and
with the sequences of the third primer and fourth primer being selected such that the third primer can hybridize specifically, under defined third conditions, with a sequence which is complementary to the first constituent segment of the first primer, and the fourth primer can hybridize specifically, under defined third conditions, with a sequence which is complementary to the third constituent segment of the second primer.

The kit can in each case contain, for each nucleic acid A to be detected, a probe which can in each case hybridize specifically, under defined fourth conditions, with the intermediate segment i or the intermediate segment i′ which is complementary thereto. The probes can be immobilized, in particular in a specific arrangement, for example on a chip.

The first constituent segments of the first primers contained in the kit and/or the third constituent segments of the second primers contained in the kit are preferably identical. This makes it possible to use standardized third and/or fourth primers for all the nucleic acids A to be detected.

The sequences of the intermediate segments i are preferably selected such that the fourth conditions are identical for all the intermediate segments i or the intermediate segments i′ which are complementary thereto. This enables all the intermediate segments i, or intermediate segments i′ which are complementary thereto, of the third primer extension products to bind simultaneously to the respective probe. The choice of the sequences enables the method to be carried out in a simplified and accelerated manner.

The kit can furthermore contain an arrangement of electrodes, with in each case one probe being immobilized on, or in the immediate vicinity of, each electrode in the arrangement. In this connection, the probes are immobilized such that it is possible to unambiguously assign each electrode to a probe. The electrode arrangement can consist of electrodes which are arranged on a surface. The arrangement of electrodes can be an electrode chip.

Instead of the first primer pair, the kit can contain specifications for the sequences of the first constituent segment, the third constituent segment and the intermediate segment i or for the sequences which are in each case complementary thereto. Using these specifications, the user of the kit can himself prepare, or have prepared for him, the first primer pair for any arbitrary nucleic acids A to be detected. In that which follows, exemplary embodiments of the invention are explained in more detail with the aid of the drawing. In this drawing:

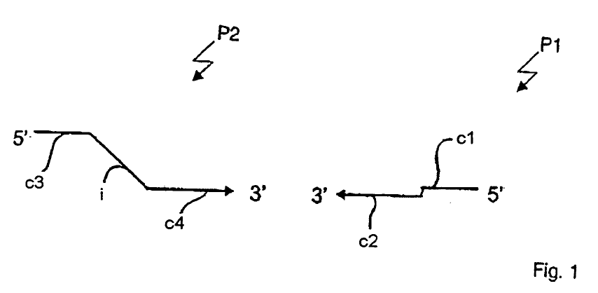

FIG. 1 shows a diagram of a first primer (P1) and a second primer (P2),

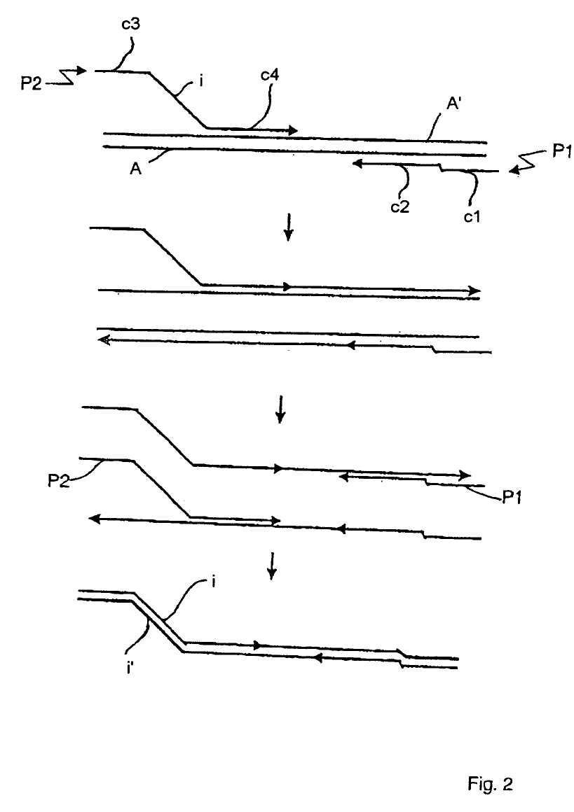

FIG. 2 shows a diagram of a first primer extension reaction and a second primer extension reaction,

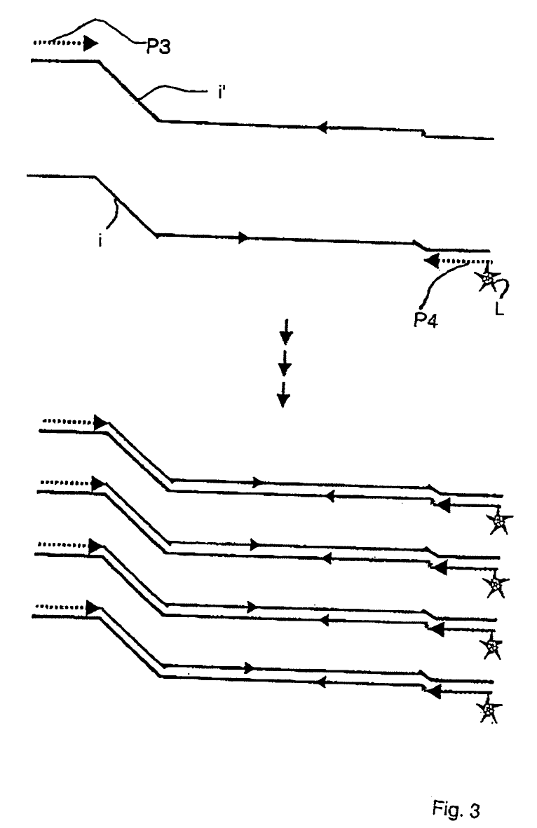

FIG. 3 shows a diagram of a PCR, and

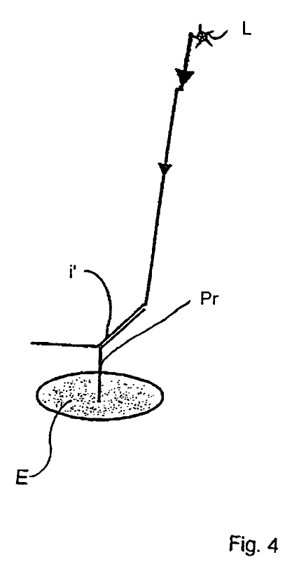

FIG. 4 shows a diagram of a primer extension product which is hybridized with an immobilized probe.

The first primer P1 depicted in FIG. 1 consists of a 5′-terminal first constituent segment c1 and a 3′-terminal second constituent segment c2. The second primer P2 consists of a 5′-terminal third constituent segment c3 and a 3′-terminal fourth constituent segment c4. The intermediate segment i is located between the third constituent segment c3 and the fourth constituent segment c4. The second constituent segment c2 is complementary to a predetermined first segment in a nucleic acid A to be detected. The fourth constituent segment c4 is complementary to a predetermined second segment of the nucleic acid A′ which is complementary to the nucleic acid A to be detected. The first constituent segment c1 and the third constituent segment c3 are preferably not complementary either to a segment of the nucleic acid A or to the nucleic acid A′ which is complementary thereto.

The two upper images in FIG. 2 depict first primer extension reactions. For these, the nucleic acid A which is present as a double strand together with the nucleic acid A′ is first of all denatured, i.e. made single-stranded, for example by an increase in the temperature. The second constituent segment c2 then hybridizes with the first segment of the nucleic acid A to be detected and the fourth constituent segment c4 hybridizes with the second segment of the nucleic acid A′ which is complementary to the nucleic acid A. In the first primer extension reaction, the first primer P1 and the second primer P2 are in each case extended at least far enough for the respective other primer P2 or P1 to be able to bind to a first primer extension product which is formed in this reaction. In a second primer extension reaction, which is subsequently carried out and which is depicted in the two lower images in FIG. 2, the in each case first primer extension product serves as a template. Two primer extension products are formed, each of which exhibits a sequence which is complementary to the first primer P1 and a sequence which is complementary to the second primer P2. The method can also be carried out using a nucleic acid A to be detected which is initially present without the nucleic acid A′ which is complementary thereto. The second primer P2 could then hybridize for the first time with the first primer extension product instead of with the complementary nucleic acid A′.

The upper image in FIG. 3 shows second primer extension products which are formed from the extension of the first primers P1 and the second primers P2. In each case a third primer P3 and a fourth primer P4, which are provided with a labeling substance or a label, hybridize by their 5′ ends. Implementing a PCR results in a large number of third primer extension products of the third and fourth primers P3 and P4. This is depicted diagrammatically in the lower image in FIG. 3. The intermediate segment i, or an intermediate segment i′ which is complementary thereto, is amplified in the PCR.

FIG. 4 depicts the hybridization of an intermediate segment i′, which is complementary to the intermediate segment i, with a sequence, which is complementary thereto, of a probe Pr which is immobilized on an electrode E. As a result of the hybridization, the label L is brought so close to the electrode E that, in this position, it can be detected by means of a change in an electrical property. The label L can be a redox-active substance, such as osmium tetroxide, which can be reduced or oxidized at the electrode. The redox signal of the label L, which signal can be measured by means of the electrode E, consequently indicates the causative presence of the nucleic acid A.

1. A method for detecting different nucleic acids A in parallel, comprising the following steps:
a) providing in each case one first primer pair which is suitable for carrying out a PCR together with one of the nucleic acids A and which contains a first primer (P1) and a second primer (P2),
with the first primer (P1) exhibiting a 5′ terminal first constituent segment (c1) and a 3′ terminal second constituent segment (c2) and the second primer (P2) exhibiting a 5′ terminal third constituent segment (c3) and a 3′ terminal fourth constituent segment (c4),
with the sequences of the second constituent segment (c2) and the fourth constituent segment (c4) being selected such that the second constituent segment (c2) can hybridize specifically, under defined first conditions, with a predetermined first segment of the one of the nucleic acids A, and be enzymically extended, and the fourth constituent segment (c4) can hybridize specifically, under defined second conditions, with a predetermined second segment of a nucleic acid A′ which is complementary to the one of the nucleic acids A, and be enzymically extended, and
with in each case an intermediate segment i, which connects the first constituent segment (c1) to the second constituent segment (c2) and is specific for the second constituent segment (c2), or an intermediate segment i, which connects the third constituent segment (c3) to the fourth constituent segment (c4) and is specific for the fourth constituent segment (c4), being provided,
with the first (P1) or second primers (P2) of the first primer pairs in each case differing in the intermediate segment i and in the second constituent segment (c2) or fourth constituent segment (c4) which is arranged in connection thereto, with each of the second (c2) or fourth constituent segments (c4) being specific for precisely one of the nucleic acids A,
b) bringing the different nucleic acids A, or the nucleic acids A′ which are complementary thereto, into contact with the first primer pairs in a solution and carrying out a first primer extension reaction in which the first primers (P1) are extended, under the first conditions, or the second primers (P2) are extended, under the second conditions, at least once and at least so far that the respective other primers (P2, P1) of the first primer pairs are able to bind specifically, under the first or second conditions which are required for their specific hybridization, to in each case one first primer extension product which is formed in this connection,
c) carrying out a second primer extension reaction in which the first primer extension products in each case serve as a template and the respective second (P2) or first primers (P1) are extended, under the first or second conditions which are required for their specific hybridization with the respective first primer extension products, with the formation of in each case one second primer extension product,
d) providing in each case one second primer pair which in each case contains a third primer (P3) and a fourth primer (P4) and which is suitable for carrying out a PCR together with the respective second primer extension products,
with the sequences of the third primer (P3) and fourth primer (P4) being in each case selected such that the third primer (P3) can in each case hybridize specifically, under defined third conditions, with a sequence which is complementary to the first constituent segment (c1), and be enzymically extended, and the fourth primer (P4) can in each case hybridize specifically, under defined third conditions, with a sequence which is complementary to the third constituent segment (c3) and be enzymically extended,
e) bringing the second primer extension products into contact with the respective second primer pairs and carrying out a PCR, with in each case the intermediate segment i, or an intermediate segment i′ which is complementary thereto, being amplified with the formation of third primer extension products,
f) providing in each case one immobilized probe (Pr) for each nucleic acid A to be detected, with the probe (Pr) being in each case able to hybridize specifically, under defined fourth conditions, with one of the intermediate segments i or one of the intermediate segments i′ which are complementary thereto,
g) bringing the probes (Pr) into contact with the third primer extension products under the fourth conditions, and
h) detecting the third primer extension products which bind, or are bound, to the probes (Pr).

2. The method as claimed in claim 1, wherein the first primer extension reaction and the second primer extension reaction are carried out as PCRs.

3. The method as claimed in claim 1, wherein the first primer extension reaction and/or the second primer extension reaction and/or the PCR(s) is/are carried out under hot start conditions.

4. The method as claimed in claim 1, wherein the first primer extension reaction is carried out, under the first conditions, and/or the second primer extension reaction is carried out, under the second conditions, at most 10 times, preferably at most 5 times, in particular at most 2 times.

5. The method as claimed in claim 1, wherein the sequences of the first constituent segment (c1) and third constituent segment (c3) are selected such that the third conditions can be so stringent that the second constituent segment (c2) does not significantly hybridize, under the third conditions, with the first segment of the one of the nucleic acids A and the fourth constituent segment (c4) does not significantly hybridize, under the third conditions, with the second segment of the nucleic acid A′ which is complementary to the one of the nucleic acids A.

6. The method as claimed in claim 1, wherein the sequences and concentrations of the first (P1), second (P2), third (P3) and fourth primers (P4) are selected such that the specific annealing temperatures of the third primer (P3), which hybridizes with the sequence which is complementary to the first constituent segment (c1), and of the fourth primer (P4), which hybridizes with the sequence which is complementary to the third constituent segment (c3), are in each case at least 5° C. higher than the in each case higher annealing temperatures of the second constituent segment (c2), which hybridizes with the first segment of one of the nucleic acids A, and of the fourth constituent segment (c4), which hybridizes with the second segment of the complementary nucleic acid A′.

7. The method as claimed in claim 1, wherein step e is carried out in the solution.

8. The method as claimed in claim 1, wherein at least steps a to e, in particular steps a to h, are carried out in a closed vessel which is not opened between the steps.

9. The method as claimed in claim 1, wherein the concentration, in the solution, of the first or second primer (P1, P2) containing the intermediate segment i is selected to be so low that this primer (P1, P2) does not significantly inhibit a hybridization of the probe (Pr) with the respective intermediate segment i, or the intermediate segment i′ which is complementary thereto, of the third primer extension products in step g.

10. The method as claimed in claim 1, wherein the concentration, in the solution, of the in each case first primer pair is set to be from 0.001 to 0.1 μmol/l.

11. The method as claimed in claim 1, wherein the ratio of the concentrations of the in each case first primer pair to the in each case second primer pair is less than 1:10, preferably less than 1:100, particularly preferably less than 1:1000.

12. The method as claimed in claim 1, wherein the second primer pairs are added to the solution prior to the first primer extension reaction.

13. The method as claimed in claim 1, wherein, in step e, in each case the third primer (P3) or the fourth primer (P4) is extended more frequently than is the respective other primer (P4, P3) of the respective second primer pairs.

14. The method as claimed in claim 1, wherein, in the second primer pair which is provided in step d, the third primer (P3) or the fourth primer (P4) is present in excess as compared with the respective other primer (P4, P3) which is present therein.

15. The method as claimed in claim 1, wherein a multiplicity of first primer pairs whose first primers (P1) exhibit an in each case identical or almost identical first constituent segment (c1) and/or whose second primers (P2) exhibit an in each case identical or almost identical third constituent segment (c3), and whose second constituent segment (c2) or fourth constituent segment (c4) is in each case specific for precisely one of the nucleic acids A, is added to the solution.

16. The method as claimed in claim 1, wherein the sequences of the first (P1), second (P2), third (P3) and fourth primers (P4) are selected such that they do not form any primer dimers and/or do not hybridize with themselves or with each other in the method.

17. The method as claimed in claim 1, wherein the sequences of the intermediate segments i are selected such that neither they themselves, nor the intermediate segments i′ which are complementary thereto, hybridize, in the method, with themselves or with the first (c1), second (c2), third (c3) or fourth constituent segments (c4) or their complementary sequences.

18. The method as claimed in claim 1, wherein the sequences of the intermediate segments i are selected such that hybrids of the intermediate segments i with nucleic acids which were in each case completely complementary thereto would have melting temperatures which are essentially identical, lying, in particular, in a temperature range of 5° C.

19. The method as claimed in claim 1, wherein, for specifically detecting one of the nucleic acids A in the presence of another nucleic acid which only differs from the one of the nucleic acids A in one first base which is contained in the one of the nucleic acids A, the sequences of the first (P1) or second primers (P2) are selected such that the respective base of the second (c2) or fourth constituent segment (c4), which base is complementary to the first base or to a second base, which is complementary thereto, of a complementary nucleic acid A′. is located at the 3′ end, or in the vicinity of the 3′ end, of the in each case first (P1) or second primer (P2).

20. The method as claimed in claim 1, wherein the second (c2) or fourth constituent segments (c4) contain a base which is not complementary to a third base, which corresponds to it in its position, in the first segment of the one of the nucleic acids A or in the second segment of the nucleic acid A′.

21. The method as claimed in claim 1, wherein the respective sequences of the first (P1), second (P2), third (P3) and fourth primers (P4), and of the probe (Pr), are selected such that in each case the first, in each case the second, in each case the third and/or in each case the fourth conditions for detecting the different nucleic acids A are identical.

22. The method as claimed in claim 1, wherein the probe (Pr) is in each case immobilized on an electrode (E) or in its immediate vicinity.

23. The method as claimed in claim 1, wherein the detection in step h is effected by detecting a change in a fluorescence-optical property or a change, which is determined by the hybridization, in an electrical property at the electrode (E).

24. The method as claimed in claim 23, wherein a change in a redox property, in particular in association with the oxidation of guanine or adenine residues of the third primer extension products, in an impedance or in a conductivity is measured, as the change in the electrical property, using the electrode (E).

25. The method as claimed in claim 1, wherein the third primer (P3) and/or the fourth primer (P4) exhibits a label which can be detected, in particular, fluorescence-optically or electrically or electrochemically by means of the electrode (E) and which is preferably redox-active.

26. The method as claimed in claim 25, wherein the label exhibits a specific affinity molecule, an osmium complex, a nanogold particle, a cysteine, ferrocenyl, daunomycin, benzoquinone, naphthoquinone, anthraquinone or p aminophenol group, a dye, in particular indophenol, thiazine or phenazine, or a fluorescent dye, in particular 6 FAM, HEX, TET, Cy3, Cy5, IRDye™700, IRDye™800, Biodipy, fluorescein, Joe, Rox, TAMRA or Texas Red.

27. The method as claimed in claim 25, wherein the label is an affinity molecule and it is detected using a counter molecule which specifically binds the affinity molecule, with the counter molecule being conjugated with an enzyme which can convert a substrate such that the reaction product can be specifically detected electrochemically or optically.

28. The method as claimed in claim 1, wherein use is made of a multiplicity of different probes (Pr) which are complementary to the intermediate segments i or to the intermediate segments i′ which are complementary thereto, each of which probes is bound to, or in the immediate vicinity of, a separate electrode (E).

29. The method as claimed claim 1, wherein use is made of a multiplicity of electrodes (E) which are arranged on a surface, in particular an electrode chip, so as to be individually bonded or bondable.

30. The method as claimed in claim 1, wherein an RNA is detected indirectly by transcribing it into a DNA and then detecting the DNA as nucleic acid A.

31. A kit for carrying out a method, as claimed in claim 1, for detecting a multiplicity of different nucleic acids A in parallel, with the kit comprising:
a) for each nucleic acid A to be detected, in each case one first primer pair which is suitable for carrying out a PCR together with the nucleic acid A and which contains a first primer (P1) and a second primer (P2),
with the first primer (P1) exhibiting a 5′ terminal first constituent segment (c1) and a 3′ terminal second constituent segment (c2) and the second primer (P2) exhibiting a 5′ terminal third constituent segment (c3) and a 3′ terminal fourth constituent segment (c4),
with the sequences of the second constituent segment (c2) and the fourth constituent segment (c4) being selected such that the second constituent segment (c2) can hybridize specifically, under defined first conditions, with a predetermined first segment of the nucleic acid A which is in each case to be detected, and the fourth constituent segment (c4) can hybridize specifically, under defined second conditions, with a predetermined second segment of a nucleic acid A′ which is complementary to the nucleic acid A which is in each case to be detected, and
with an intermediate segment i, which connects the first constituent segment (c1) to the second constituent segment (c2) and is specific for the second constituent segment (c2), or an intermediate segment i, which connects the third constituent segment (c3) to the fourth constituent segment (c4) and is specific for the fourth constituent segment (c4), being provided, and
b) for each nucleic acid A to be detected, in each case a second primer pair containing a third primer (P3) and a fourth primer (P4), which pair is suitable, together with a primer extension product which can be generated, using the first (P1) and second (P2) primers, when the nucleic acid A which is in each case to be detected is present, for carrying out a PCR, and
with the sequences of the third primer (P3) and fourth primer (P4) being selected such that the third primer (P3) can hybridize specifically, under defined third conditions, with a sequence which is complementary to the first constituent segment (c1) of the first primer, and the fourth primer (P4) can hybridize specifically, under defined third conditions, with a sequence which is complementary to the third constituent segment (c3) of the second primer,
with the kit in each case containing, for each nucleic acid A to be detected, a probe (Pr) which can in each case hybridize specifically, under defined fourth conditions, with the intermediate segment i or the intermediate segment i′ which is complementary thereto.

32. The kit as claimed in claim 31, wherein the probes (Pr) are immobilized.

33. The kit as claimed in claim 31, wherein the first constituent segments (c1) of the first primers (P1) contained in the kit are identical and/or the third constituent segments (c3) of the second primers (P2) contained in the kit are identical.

34. The kit as claimed in claim 31, wherein the sequences of the intermediate segments i are selected such that the fourth conditions are identical for all the intermediate segments i or the intermediate segments i′ which are complementary thereto.

35. The kit as claimed in claim 31, which contains an arrangement of electrodes (E), with in each case one probe (Pr) being immobilized on, or in the immediate vicinity of, each electrode (E) in the arrangement.

36. The kit as claimed in claim 35, wherein the arrangement of electrodes (E) is an electrode chip.

37. The kit as claimed in claim 31 which contains, instead of the first primer pair, specifications for the sequences of the first constituent segment (c1), the third constituent segment (c3) and the intermediate segment i or for the sequences which are in each case complementary thereto.
